(19) United States
(12) Patent Application Publication
(10) Pub. No.: US 2008/0110782 A1
(43) Pub. Date: May 15, 2008

(54) Array of packages having relative size indicators

(76) Inventors: Brian Kenneth Burgdorf, Norwood, OH (US); Gabriela Valerio Gonzalez, Cincinnati, OH (US); Jessica Lee Hansman, Cincinnati, OH (US); Jean Marie Knuth, Cincinnati, OH (US); Signe Christina Larson, Cincinnati, OH (US); Eduardo Enrique Souchon, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/595,709

(22) Filed: Nov. 10, 2006

(51) Int. Cl. A61F 13/15; B65D 81/26; B65D 85/00

(52) U.S. Cl. 206/438; 206/204; 206/459.5

(57) ABSTRACT

An array of packages wherein each package has relative size indicators is provided. The relative size indicators can be any indicator suitable for visually communicating to a consumer, the relative size of the absorbent articles contained in a package, in relation to other absorbent articles contained in other packages within an array of packages. The packages can comprise an interior surface and an exterior surface, the interior surface defining an interior space. Absorbent articles can be disposed within the interior space and the exterior surface can include relative size indicators.

### FIELD OF THE INVENTION

This invention relates to arrays of packages, particularly wherein the packages contain absorbent articles.

### BACKGROUND OF THE INVENTION

Absorbent articles can include sanitary napkins, liners, tampons, interlabial products, and incontinence articles. There are many variations of sizes, shapes, and conformations of absorbent articles, even within a particular brand or category. Because of this diversity, consumers can have difficulty differentiating between the various types of absorbent articles, even once they have chosen a particular brand or category.

In addition, absorbent articles such as sanitary napkins have generally been contained in printed or marked packages. The markings on the packages have typically been used to attract the consumer's attention to the absorbent article, or to communicate a property of the article to the consumer. At times however, these communications in combination with the overall packaging of the article have failed to communicate the desired message to the consumer, or have misdirected or confused the consumer as to the type of absorbent article in the package. Typically, the package can include labeling, instructions, and/or a picture of the absorbent article. Nevertheless, the consumer may still be unable to determine quickly the size of the absorbent article. In some instances, the consumer may be unable to determine the size of the absorbent article by the package exterior, or may have a limited amount of time to make a selection of a given absorbent article. In others, language difficulties can contribute to improper selection and usage.

Absorbent article containing packages with size indicators disposed thereon are known in the art. The size indicators are generally in the form of multiple drawings of absorbent articles, arranged in a manner consistent with the suggested mode of use. For example, the multiple drawings could be organized in a row from low absorbency to high. The drawing that corresponds to the absorbent article contained in the package, is usually made to stand out from the other drawings. This is usually done by modifying its appearance from the other drawings, such as by it having a color different from the other drawings. While this method does indicate to the consumer the absorbent article's position in the suggested mode of use, and even in some instances its size in comparison to other absorbent articles shown in the drawings, the method does not help prevent consumer confusion as noted above. For example, the use of multiple drawings on a single package, does not help a consumer differentiate the sizes of absorbent articles contained in separate packages, irrespective of the size of the packages.

It would be desirable to have an array of packages wherein each package has visual indicators located thereon, wherein the visual indicators convey to a consumer the size of the absorbent articles contained therein, in relation to other packages within the array.

### SUMMARY OF THE INVENTION

An array of packages is provided comprising a first package having a size, said first package having disposed therein a first absorbent article having a size; a first relative size indicator disposed on said first package, said first relative size indicator being an image having a first predetermined maximum dimension; a second package having a size, said second package having disposed therein a second absorbent article having a size different from the size of said first absorbent article; a second relative size indicator disposed on said second package, said second relative size indicator being an image having a second predetermined maximum dimension; wherein said first predetermined maximum dimension of said first relative size indicator differs from said second predetermined maximum dimension of said second relative size indicator in a relationship corresponding to the relative difference between the size of said first absorbent article and the size of said second absorbent article.

An array of packages is provided which comprises a first package having a size, said first package having disposed therein a first sanitary napkin having a size; a first relative size indicator disposed on said first package, said first relative size indicator being an image having a first predetermined maximum dimension; a second package having a size, said second package having disposed therein a second sanitary napkin having a size different from the size of said first sanitary napkin; a second relative size indicator disposed on said second package, said second relative size indicator being an image having a second predetermined maximum dimension; wherein said first predetermined maximum dimension of said first relative size indicator differs from said second predetermined maximum dimension of said second relative size indicator in a relationship corresponding to the relative difference between the size of said first sanitary napkin and the size of said second sanitary napkin.

### BRIEF DESCRIPTION OF THE DRAWINGS

FIG. 1 is a perspective view of one embodiment of a relative size indicator of the present invention.

FIG. 2 is a perspective view of one embodiment of the present invention.

FIG. 3 is a perspective view of one embodiment of the present invention.

FIG. 4 is a perspective view of one embodiment of the present invention.

FIG. 5 is a perspective view of one embodiment of the present invention.

FIG. 6 is a perspective view of one embodiment of the present invention.

### DETAILED DESCRIPTION OF THE INVENTION

The present invention relates to an array of packages having relative size indicators located thereon. Each package within the array has absorbent articles disposed therein. The relative size indicator located on each package within the array communicates to the consumer the relative size of the absorbent articles located therein, in relation to the size of other absorbent articles contained in other packages within the array. Therefore, by viewing an array of packages with each package having relative size indicators, a consumer is able to determine the relative size of an absorbent article in one package, in relation to that of other absorbent articles contained in other packages within the array.

As used herein, by array of packages is meant more than one package in a line-up of consumer products. The consumer products can be, and usually are, branded products in which the packages in the array of packages exhibit a common brand name. for example, the ALWAYS® sanitary napkin brand includes an array of different products, such as Ultra, Maxi, Regular, Super, Overnight, and the like, that are usually marketed as an array of packages at a point of sale. In one embodiment, an array of packages is a plurality of packages, each labeled with the same brand name.

As used herein, the term “absorbent article” refers to devices that absorb and/or contain a substance, such as, body exudates. A typical absorbent article can be placed against or in proximity to the body of a wearer to absorb and contain various body exudates. Examples, of absorbent articles include feminine hygiene articles, and baby diapers. The size of an absorbent article is determined by using whatever standard method of measurement is utilized by one of ordinary skill in the art when measuring the type of absorbent article. For example, the size of a sanitary napkin may be determined by measuring the total area of the sanitary napkin when it is spread open and laid out flat against a planar surface, or by measuring its longest linear dimension.

As used herein, the term “feminine hygiene article” refers to articles such as, disposable absorbent articles that can be worn by women for menstrual and/or light incontinence control, such as, sanitary napkins, tampons, interlabial products, incontinence articles, and pantiliners. As used herein, a feminine hygiene article can include any associated wrapping or applicator that typically can be associated with the feminine hygiene article. For example, a feminine hygiene article can be a tampon that may or may not include an applicator and/or can be a sanitary napkin that may or may not include a wrapper, such as, a wrapper that individually encloses a sanitary napkin.

As used herein, the term “sanitary napkin” refers to an absorbent structure such as, for example an absorbent mass that can be placed against or in close proximity to the pudendal region for absorbing menses or urine. Examples of sanitary napkins are those manufactured by The Procter & Gamble Company of Cincinnati, Ohio as: ALWAYS® Pantiliners with DriWeave® manufactured according to U.S. Pat. Nos. 4,324,246; 4,463,045; and 6,004,893; ALWAYS® Ultrathin Slender Maxi with Wings manufactured according to U.S. Pat. Nos. 4,342,314, 4,463,045, 4,556,146, B1 4,589,876, 4,687,478, 4,950,264, 5,009,653, 5,267,992, and Re. 32,649; ALWAYS® Regular Maxi; ALWAYS® Ultra Maxi with Wings; ALWAYS® Maxi with Wings; ALWAYS® Ultra Long Maxi with Wings; ALWAYS® Long Super Maxi with Wings; and ALWAYS® Overnight Maxi with Wings, each aforesaid publication being incorporated by reference herein.

As used herein, the term “image” refers to any type of mark, figure, geometric shape, picture, illustration, symbol, icon, pattern, or any other indicia on a package within an array of packages having a purpose of communicating to a consumer something about the absorbent articles contained in a package. The image can be made in any suitable manner, such as, by printing, embossing, transferring, stamping, debossing, etching, or any combination thereof.

The relative size indicators of the present invention can be any image suitable for visually communicating to a consumer the relative size of the absorbent articles contained in a package, in relation to other absorbent articles contained in other packages within an array of packages. For example, in a package of absorbent articles the relative size indicator can be any image such as a graphic representation of the enclosed absorbent article. In certain embodiments there may be more than one relative size indicator on a package.

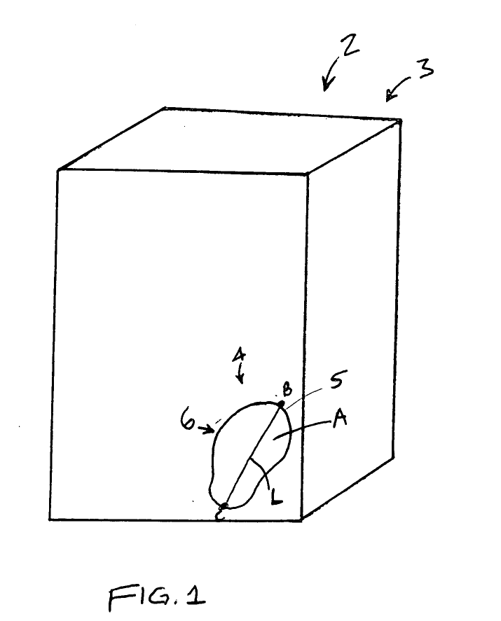

A shown in FIG. 1 the package 2 has an exterior surface 3, wherein the term “exterior surface” as used herein refers to the surface of the package, which is exposed to the environment. The relative size indicator 4 can be placed on the exterior surface 3 of the package 2. The relative size indicator 4 can have a perimeter 5 which can be an outer boundary of an image 6, defining a total area “A” for the image 6. In certain embodiments the PMD of the relative size indicator 4 can be the total area “A” of the image 6. The PMD of the relative size indicator 4 may also be the longest linear dimension “L” as measured from any two points B, C on the perimeter 5 of the image 6. In certain embodiments, the package can include one or more absorbent articles disposed within the package and can also include one or more relative size indicators.

In certain embodiments, the absorbent articles contained within the package can be viewed by the consumer, such as, for example, through a window or other opening on the package. The absorbent articles can be viewed by the consumer at any suitable location on the package, such as, for example, from the front of the package and/or from any other side typically viewed by the consumer. In certain embodiments, a window can be situated close to the location on the package of the relative size indicators.

The relative size indicator can be located at any suitable location on the package. In certain embodiments, the relative size indicator can be located on the exterior surface of the package. In certain embodiments, the relative size indicator can be located on a secondary packaging enclosing the package. Alternatively, or in addition, the relative size indicator can be located somewhere other than on the package. For example, in certain embodiments, the relative size indicator can be located on the wrapper of an absorbent article such as a tampon or sanitary napkin.

A package can be any suitable size or shape. In certain embodiments, the package can be made of inflexible materials, such as, for example, cardboard, paperboard, cartonboard, chipboard, plywood, SBS, metal, plastic, paper, card stock, fabric, ceramic, polymer, natural or synthetic fibers, webs, mesh, screen, wood, composite, mixtures or combinations thereof, or any other suitable material. Alternatively, or in addition, the package can be made of a flexible material, such as, blown or cast film in a blend of low density polyethylene and linear low density polyethylene, metallocenes, ethylene vinyl acetate, surlyn, polyethylene terephthalate, biaxally oriented polypropylene, nylon, combinations thereof, or any other suitable material.

In certain embodiments the size of a package is determined by the interior volume of the package. Additionally, the size of a package may be determined by the measurement of the display side's total area, wherein the display side of a package is the side that is intended to be viewed by a customer when the package is on a retail shelf. The display side of a package is the side that is generally used to communicate information regarding the absorbent article contained therein to the consumer. The size of the display side can also confuse consumers as to the size of the absorbent articles the package contains, in relation to the size of other absorbent articles in packages. For example, the consumer can believe the larger the display side of the package, the larger the absorbent article contained therein. However, this is not always the case, as a larger size package might actually contain smaller sized absorbent articles, in relation to the absorbent articles contained in a smaller sized package.

The package can include at least one window, such as, a region of transparency. As used herein, “transparency” means having the property of transmitting light without substantial scattering so that items lying beyond can be visible to a person. The window can comprise a transparent covering, such as, for example, a transparent film. In addition, or alternatively, the window can comprise an opening in the package that does not include a covering. In certain embodiments, the window can comprise a transparent covering that can be clear, translucent, tinted and/or opaque. The transparent covering can be tinted in any suitable color or combination of colors. The transparent covering can also, or alternatively, be printed and/or embossed. In certain embodiments, the window can be images that depict and/or give the perception of a window, such as, images that depict a region of transparency and/or an opening.

The window can be disposed on at least one side of the package, such as, the front, the back, and/or sides. In certain embodiments, the window can be disposed on more than one side. The window can be at any location on the side and can comprise any appropriate amount of the package side, such as, for example, from about 5% to about 95% of the side, such as, for example, about 5% to about 75%, about 5% to about 50%, about 5% to about 25%, or any other amount of the side. In certain embodiments, absorbent articles with a relative size indicator disposed thereon can be positioned adjacent the window such that at least a portion of the absorbent articles and/or relative size indicator, can be visible through the window. In certain embodiments the wrapper of an absorbent article with a relative size indicator disposed thereon can be positioned adjacent the window such that at least a portion of the wrapper of the absorbent article and/or relative size indicator, can be visible through the window.

Any absorbent article or combination of absorbent articles can be disposed in the package. In certain embodiments, the absorbent articles can be individually wrapped prior to being disposed within the package. In certain embodiments, the package can contain a single absorbent article.

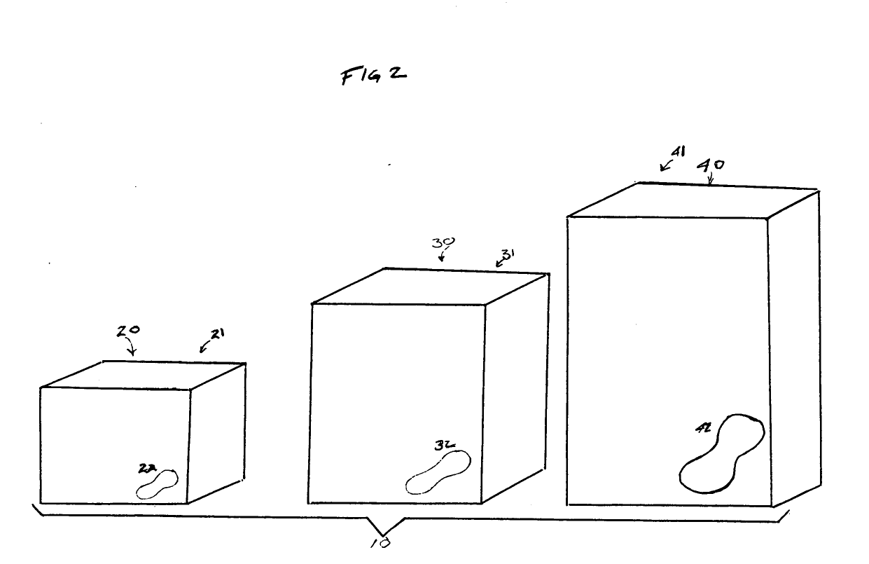

FIG. 2 shows an example of an array of packages 10, with each package 20, 30, 40 in the array 10 differing in size, in both the interior volume of the package and the measurement of the display side's total area, and each having disposed on one face a relative size indicator 22, 32, 42 respectively. The packages 20, 30, 40 have an exterior surface 21, 31, 41. The relative size indicators 22, 32, 42 can be shown on the exterior surfaces 21, 31, 41 of the packages 20, 30, 40. In this example, the packages 20, 30, 40 within the array 10 have relative size indicators 22, 32, 42 that are images of sanitary napkins and correspond to the relative size of the sanitary napkin enclosed. Each of the relative size indicators 22, 32, 42 differs in PMD in this embodiment by both total area and longest linear dimension, specifically with relative size indicator 42 having a greater PMD than relative size indicators 32 and 22, and relative size indicator 32 having a greater PMD than relative size indicator 22. The differing PMD's of the relative size indicators 22, 32, 42 visually communicates to the consumer the relative size of the absorbent articles contained in the packages 20, 30, 40 in relation to the absorbent articles in other packages 20, 30, 40 of the array 10. In this example, the relative size indicators 22, 32, 42 communicate to the consumer that as the PMD of the relative size indicators 22, 32, 42 increases, the size of the absorbent article contained within the respective package 20, 30, 40, also increases.

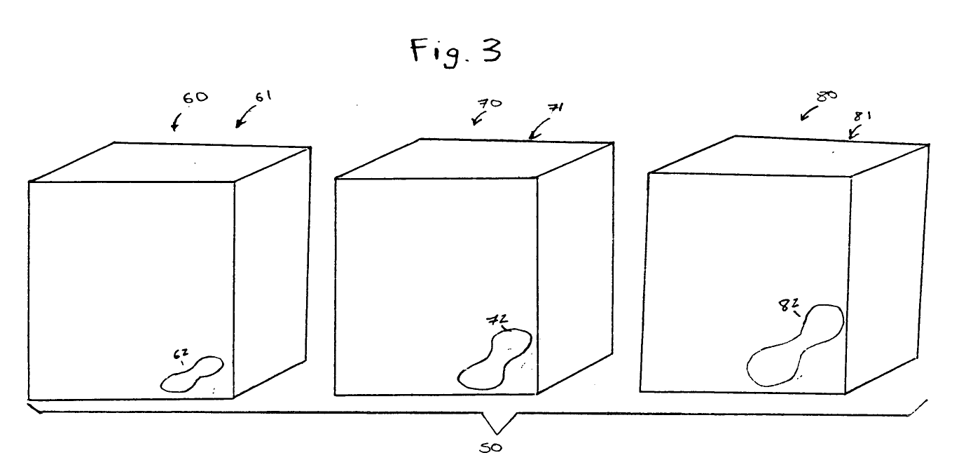

FIG. 3 shows an example of an array of packages 50, with each package 60, 70, 80 in the array 50 having the same or similar size, in both the interior volume of the package and the measurement of the display side's total area and each having a relative size indicator 62, 72, 82. The packages 60, 70, 80 have an exterior surface 61, 71, 81. The relative size indicators 62, 72, 82 can be placed on the exterior surfaces 61, 71, 81 of the packages 60, 70, 80. In this example, the relative size indicators 62, 72, 82 communicate to the consumer that as the PMD of the relative size indicators 62, 72, 82 increases, the size of the absorbent article contained within the respective package 60, 70, 80 also increases. In this embodiment, consumers presented with this array of packaging can readily identify which of the identically-sized packages have the larger or smaller absorbent articles therein.

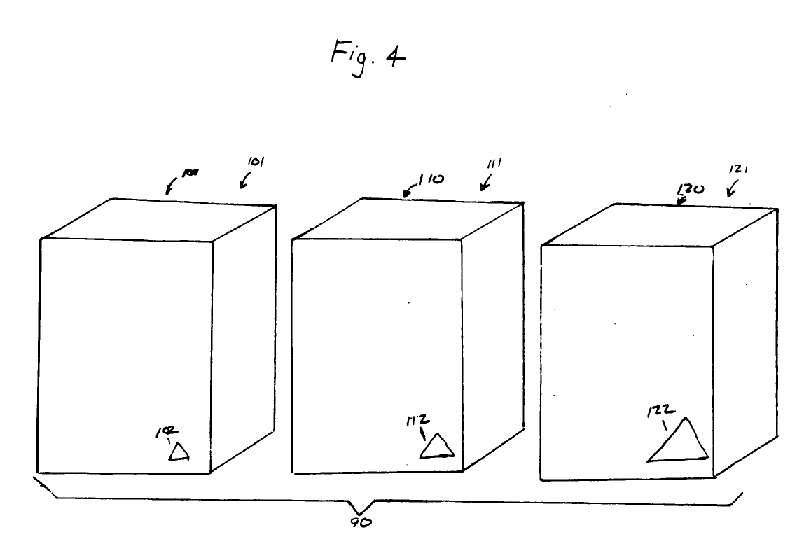

As shown in FIG. 4 in certain embodiments, the relative size indicator 102, 112, 122 can communicate to a consumer the relative sizes of the absorbent articles, contained within each package 100, 110, 120 of the array 90, without the relative size indicators 102, 112, 122 having to be graphical representations of the absorbent article contained in each package 100, 110, 120.

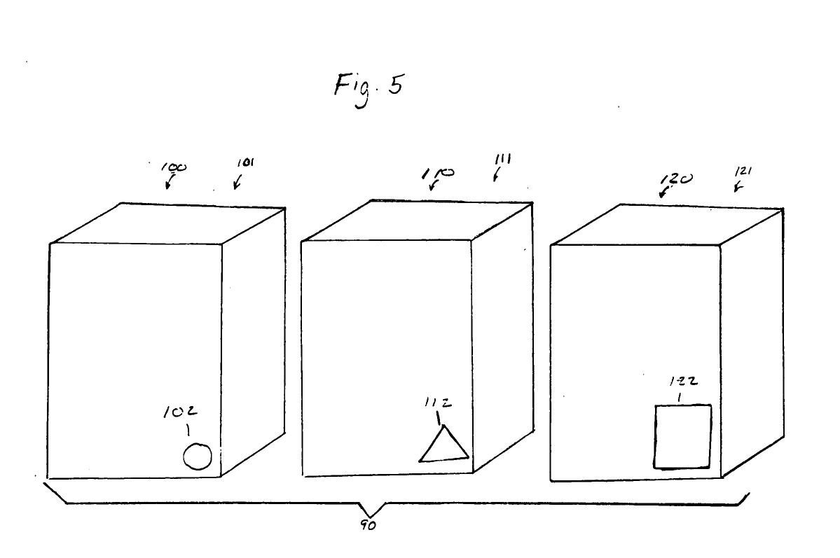

FIG. 5 shows that in certain embodiments, the relative size indicators 102, 112, 122 on packages 100, 110, 120 in the array 90 can communicate to a consumer the relative sizes of the absorbent articles through the use of differing images, without the relative size indicators 102, 112, 122 having to be graphical representations of the absorbent article contained in each package 100, 110, 120.

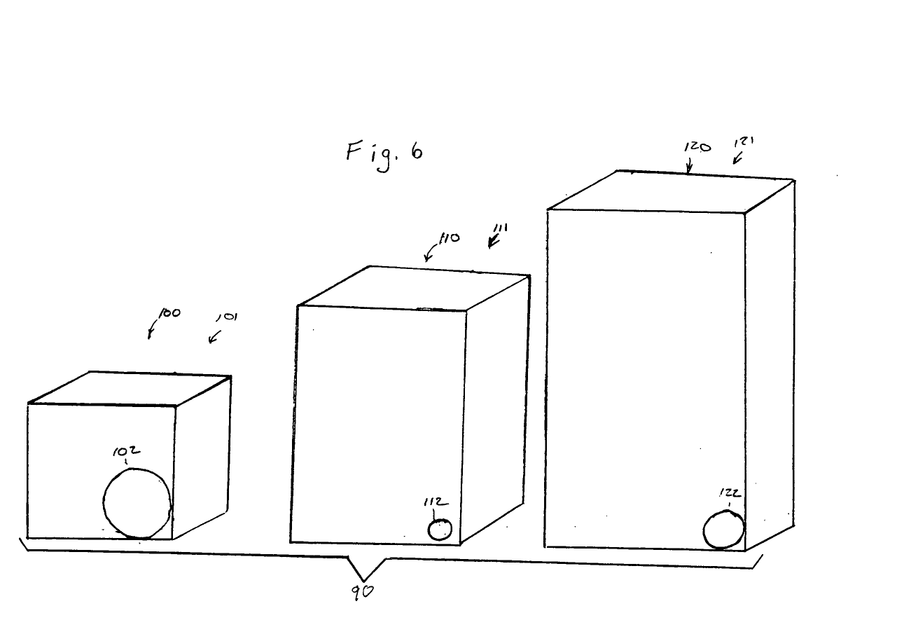

FIG. 6 shows that in certain embodiments the PMD of the relative size indicator 102, 112, 122 does not correspond to the size of the packages 100, 110, 120 but rather to the relative size of the absorbent articles contained in each package 100, 110, 120. For example, the relative size indicator 122 of the largest package 120 has a smaller PMD in comparison to the relative size indicator 102 on the smallest package 100. If used in an array of packaged absorbent articles, this visually communicates to a consumer that the absorbent articles contained in package 120, are smaller in size in relation to the absorbent articles contained in package 100, even though package 120 is larger in comparison to package 100.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of packages, said array comprising:
a first package having a size, said first package having disposed therein a first absorbent article having a size;
a first relative size indicator disposed on said first package, said first relative size indicator being an image having a first predetermined maximum dimension;
a second package having a size, said second package having disposed therein a second absorbent article having a size different from the size of said first absorbent article;
a second relative size indicator disposed on said second package, said second relative size indicator being an image having a second predetermined maximum dimension;
wherein said first predetermined maximum dimension of said first relative size indicator differs from said second predetermined maximum dimension of said second relative size indicator in a relationship corresponding to the relative difference between the size of said first absorbent article and the size of said second absorbent article.

2. The array of claim 1 wherein there is more than one first or second relative size indicator disposed on the first or second package.

3. The array of claim 1 wherein the first and second relative size indicator is an image selected from the group consisting of a mark, figure, geometric shape, picture, illustration, symbol, icon, pattern, and any combination thereof.

4. The array of claim 1 wherein the first and second relative size indicators comprise different images.

5. The array of claim 1 wherein the predetermined maximum dimension of the first or second relative size indicator is the total area of said first or second relative size indicator.

6. The array of claim 1 wherein the predetermined maximum dimension of the first or second relative size indicator is the longest linear dimension of the first or second relative size indicator.

7. The array of claim 1 wherein the size of the first package differs from the size of the second package.

8. The array of claim 7 wherein the size of the first package is less than the size of the second package.

9. The array of claim 8 wherein the size of the first absorbent article is greater than the size of the second absorbent article.

10. The array of claim 7 wherein the size of the first package is greater than the size of the second package.

11. The array of claim 10 wherein the size of the first absorbent article is less than the size of the second absorbent article.

12. The array of claim 1 wherein the absorbent article is selected from the group consisting of sanitary napkins, tampons, interlabial products, incontinence articles, liners, and baby diapers.

13. An array of packages, said array comprising:
a first package having a size, said first package having disposed therein a first sanitary napkin having a size;
a first relative size indicator disposed on said first package, said first relative size indicator being an image having a first predetermined maximum dimension;
a second package having a size, said second package having disposed therein a second sanitary napkin having a size different from the size of said first sanitary napkin;
a second relative size indicator disposed on said second package, said second relative size indicator being an image having a second predetermined maximum dimension;
wherein said first predetermined maximum dimension of said first relative size indicator differs from said second predetermined maximum dimension of said second relative size indicator in a relationship corresponding to the relative difference between the size of said first sanitary napkin and the size of said second sanitary napkin.

14. The array of claim 13 wherein there is more than one first or second relative size indicator disposed on the first or second package.

15. The array of claim 13 wherein the predetermined maximum dimension of the first or second relative size indicator is the total area of said first or second relative size indicator.

16. The array of claim 13 wherein the predetermined maximum dimension of the first or second relative size indicator is the longest linear dimension of the first or second relative size indicator.

17. The array of claim 13 wherein the size of the first package differs from the size of the second package.

18. The array of claim 17 wherein the size of the first package is less than the size of the second package.

19. The array of claim 18 wherein the size of the first sanitary napkin is greater than the size of the second sanitary napkin.
